(19) United States
(12) Patent Application Publication
(10) Pub. No.: US 2006/0069151 A1
(43) Pub. Date: Mar. 30, 2006

(54) Novel use of lycopene

(76) Inventors: Luca Barella, Basel (CH); Regina Goralczyk, Grensach-Wyhlen (DE); Ulrich Siler, Grenzach-Wyhlen (DE); Karin Wertz, Rheinfelden (DE)

Correspondence Address: Stephen M Haracz;Bryan Cave, 1290 Avenue of the Americas, New York, NY, 10104, US

(21) Appl. No.: 10/537,452

(22) PCT Filed: Dec. 4, 2003
(86) PCT No.: PCT/EP03/13665

§ 371 Date: Jun. 3, 2005

(30) Foreign Application Priority Data
Dec. 6, 2002 (EP) 02027244.9

(51) Int. Cl. A61K 31/375; A61K 31/355; A61K 31/353; A61K 31/015

(52) U.S. Cl. 514/456; 514/458; 514/474; 514/763

(57) ABSTRACT

Lycopene can be used in the primary prevention and incidence risk reduction of non-cancerous symptoms and/or pathologies (i.e., the prophylactic supplementation of the healthy subjects), in the treatment or the coadjuvant treatment of non-can-cerous symptoms and/or pathologies (i.e. the supplementation as therapy or accompanying a running therapy) and in the secondary prevention of non-cancerous symptoms and/or pahtologies (i.e., the supplementation after a successful therapy for the prevention of the relapse), which are associated with, favored by or caused by androgen signalling or which are sensitive to a reduction of androgen signalling.

The present invention relates to a novel use of lycopene. More particularly, the invention relates to the use of lycopene for prevention, incidence risk reduction, coadjuvant treatment or treatment of non-cancerous symptoms and/or pathologies, which are associated with, favored by or caused by androgen signalling, or which are sensitive to a reduction of androgen signalling. More specifically, the present invention relates to the use of lycopene in the primary prevention and incidence risk reduction of non-cancerous symptoms and/or pathologies (i.e., the prophylactic supplementation of healthy subjects), in the treatment or the coadjuvant treatment of non-cancerous symptoms and/or pathologies (i.e. the supplementation as therapy or accompanying a running therapy) and in the secondary prevention of non-cancerous symptoms and/or pathologies (i.e., the supplementation after a successful therapy for the prevention of relapse), which are associated with, favored by or caused by androgen signalling or which are sensitive to a reduction of androgen signalling.

Androgen signalling is elementary for the development and maintenance of a variety of physiological functions. In males as well as in females, androgen signalling within physiological windows is necessary, and signalling intensities outside physiological ranges lead to symptoms or pathologies, whose shape or intensity alter according to androgen signalling level.

In the prostate (within benign prostate tissue), androgen signalling is necessary for the development and maintenance of prostate tissue architecture (Janulis L. et al., Prostate (2000) 43: 195-204, Huynh H. et al., J Endocrinol (2001) 171: 109-18, Kucway R. et al J Urol (2002) 167: 2443-7). In humans, the skin is a target of androgen signalling, where hair growth and sebum secretion are under androgen control. Especially female physiology seems to be even more sensitive to altered androgen levels. An increased synthesis of androgen in the ovaries (Stein-Leventhal syndrome associated with polycystic ovary syndrome or amenorrhea) or in adrenals (due to carcinogenesis or hyperplasia), or a sensitization due to increased local metabolization within the skin, shows up clinically in dermatological symptoms like male-like hair growth (hirsutism) (Kelestimur F., J Pediatr Endocrinol Metab (2001) 14 Suppl 5:1309-15; discussion 1317), alopecia (Mulinari-Brenner F & Bergfeld W F, Dermatol Nurs (2001) 13: 269-72, 277-8) and feminine acne (Vexiau P. et al., Ann Dermatol Venereol (2002) 129: 174-8, and Gynecol Obstet Fertil (2002) 30: 11-21). Polycystic ovary syndrome (PCOS) is the most frequent androgen disorder of ovarian function. PCOS women are predisposed to infertility due to the chronic anovulation. At the same time they have been found to be profoundly insulin resistant. This baseline insulin resistance combined with the worsening effect of obesity (which may affect up to 75% of the US PCOS population), places these women at increased risk for impaired glucose tolerance and most likely diabetes. Women with PCOS tend to have elevated triglycerides (perhaps the best lipid marker of insulin resistance), and an unfavorably elevated LDL (low density lipoproteins)/HDL (high density lipoproteins) ratio. A lowered HDL-C levels appear to be the strongest lipid predictor of cardiovascular mortality in women (Legro R S, Mol Cell Endocrinol (2002) 186: 219-25, Pugeat M. et al., Horm Res (2000) 54: 322-6, Jacobs D R Jr. et al, Am J Epidemiol (1990) 131: 32-47 and Wilson P W. et al., Arteriosclerosis (1988) 8: 737-41) indicating an increased risk for cardiovascular diseases in PCOS patients.

According to the present invention, it has been found that androgen target gene expression in androgen sensitive target organ tissue, as the endpoint and result of androgen signalling, can be significantly reduced by the administration of lycopene or a combination of lycopene and vitamin E.

The present invention, therefore, in one aspect is concerned with the use of lycopene in the manufacture of a composition for the primary and secondary prevention, incidence risk reduction, coadjuvant treatment or treatment of non-cancerous symptoms and/or pathologies, which are associated with, favored by or caused by androgen signalling, or which are sensitive to a reduction of androgen signalling.

In another aspect, the present invention is concerned with a method of prevention or treatment of symptoms or pathologies associated with androgen signalling, which comprises administering to a subject (mammal or non-mammal, human or pet including birds and fish, or mammal or non-mammal farm animal) in need of such treatment for therapy or prophylaxis an effective amount of lycopene.

In still another aspect, the invention is concerned with a method of treating non-cancerous symptoms and/or pathologies sensitive to lycopene comprising administering to a mammal, mammal or non-mammal pets including birds and fish, or mammal or non-mammal farm animal in need of such treatment an amount of lycopene, wherein said amount leads to a reduction of androgen signalling.

In a preferred embodiment of the invention, lycopene is used together with vitamin E and/or vitamin C. Most preferred is a combination of lycopene, vitamin E and vitamin C. The term vitamin E as used herein includes racemic vitamin E (D,L-α-tocopherol) or natural vitamin E, as well as derivatives thereof which have biological vitamin E activity, e.g. carboxylic acid esters, such as vitamin E acetate, propionate, butyrate or succinate; and 6-hdroxy-2,5,7,8-tetramethylchroman-2-carboxylic aid (also called Trolox®) and Trolox®-lipoate. The term vitamin C as used herein includes derivatives thereof, which have biological vitamin C activity, e.g. esters and salts, such as sodium ascorbate, sodium ascorbyl phosphate, sodium ascorbyl polyphosphates and ascorbyl palmitate.

In a further embodiment of the invention, lycopene or lycopene in combination with vitamin E and/or vitamin C is used together with one or more of the following compounds:
(a) Silymarin (extract from Silybum marianum) and/or one or more derivatives thereof (silymarin dihemisuccinate sodium salt) and/or one or more of its four main components (silybin [synonymous with silibinin, and sometimes incorrectly called silybinin] and/or isosilybin and/or silydianin and/or silychristin) and/or one or more derivatives thereof (silybin-dihemisuccinate, disilybin, silybin-phosphatidylcholine complex, silybin-phosphate);
(b) Extract of Saw Palmetto (Sabal serrulata, syn. Serenoa repens) and/or one or more derivatives thereof and/or one or more of its main components being free fatty acids (lauric acid, oleic acid, myristic acid, palmitic acid and/or one or more derivatives thereof) and/or phytosterols (sitosterol, campesterol, stigmasterol, cycloartenol, sitostanol, campestanol and/or derivatives thereof (long-chain fatty acyl ester, ferrulate ester, glycosides));
(c) Genistein aglycone (4′,5,7-trihydroxyisoflavone) and/or one or more derivatives thereof (genistein glucosides, genistein sulfates, genistein glucuronides);
(d) Apigenin and/or one or more derivatives thereof;
(e) Quercetin (2-(3,4-dihydroxyphenyl)-3,5,7-trihydroxy-4H-1-benzopyrano-4-one) and/or dihydroquercetin and/or one or more derivatives thereof (quercetine glucosides, quercetin glucuronides, quercetine sulphates, methylquercetin (isohamnetin (3′-O-methylquercetin), tamarixetin(4′-O-methylquercetin));
(f) Myricetin and/or one or more derivatives thereof;
(g) Kampferol and/or one or more derivatives thereof;
(h) Resveratrol (cis-3,4′,5-trihydroxystilbene and/or trans-3,4′,5-trihydroxystilbene) and/or one or more derivatives thereof (resveratrol glucosides, resveratrol sulfates, resveratrol glucuronides);
(i) Curcumin (effects of Curcuma Longa) and/or one or more derivatives (demethoxy-curcumin, bis-demethoxycurcumin, sodium curcumionate, bis-demethylcurcumin, tetrahydrocurcumin, hexahydrocurcumin, diacteylcurcumin, triethylcurcumin) thereof and/or one or more of its main components (curcumin (diferuloylmethane), demethoxycurcumin, bisdemethoxycurcumin) and/or derivatives thereof (glucuronides, sulfates),
(j) flufenamic acid and/or one or more derivatives (esters) thereof;
(k) geldanamycin
(l) Extract of Stephania hernandifolia and/or one or more derivatives thereof and/or one or more of its components (e.g. 4-demethylhasubanonine, epistephanine) and/or derivatives thereof;
(m) Extract of Myrica rubra and/or one or more derivatives thereof and/or one or more of its components being diarylheptanoids (Quercetin, myricanone, myricanol, and myricetin) named acerogenin and their glycosides named aceroside and/or derivatives thereof;
(n) Astaxanthin ((3S,3′S)-3,3′-dihydroxy-β,β-carotene-4,4′-dione) and/or one or more isomers and/or monoesters and/or diesters, preferably esters of saturated alkanoic acids, such as acetic, propionic, palmitic, stearic, and succinic acid, mono-unsaturated fatty acids, such as oleic acid, and poly-unsaturated fatty acids, such as linolic, linoleic, docosahexaenoic, and arachidonic acid;
(o) β-Carotene and/or one or more isomers thereof;
(p) β-Cryptoxanthin ((3R)-β,β-carotene-3-ol) and/or one or more isomers or esters thereof, preferably esters of saturated alkanoic acids, such as acetic, propionic, palmitic, stearic, and succinic acid, mono-unsaturated fatty acids, such as oleic acid, and poly-unsaturated fatty acids, such as linolic, linoleic, docosahexaenoic, and arachidonic acid;
(q) (−)-Epigallocatechin gallate (EGCG) and/or (−)-epicatechin gallate (ECG) and/or one or more derivatives thereof,
(r) Lutein ((3R,3′R,6′R)-β,ε,carotene-3,3′-diol) and/or one or more isomers and/or monoesters and/or diesters, preferably esters of saturated alkanoic acids, such as acetic, propionic, palmitic, stearic, and succinic acid, mono-unsaturated fatty acids, such as oleic acid, and poly-unsaturated fatty acids, such as linolic, linoleic, docosahexaenoic, and arachidonic acid, thereof;
(s) Rhizoxin and/or one or more derivatives thereof (palmitoyl rhizoxin);
(t) Vitamin A and/or retinoic acids (all-trans retinoic acid and/or 13-cis retinoic acid and/or 9-cis retinoic acid) and/or one or more derivatives thereof (all-trans retinoic acid, 13-cis retinoic acid, all-trans retinol, 9-cis retinoic acid or 4-hydroxyphenylretinamide or retinyl esters such as all-trans retinyl acetate);
(u) Vitamin D2 or vitamin D3 or 1α,25-dihydroxyvitamin D3 or 25-hydroxyvitamin D3 or 1α,24R,25-trihydroxyvitamin D3; or 24,25-dihydroxyvitamin D3
(v) Zeaxanthin ((3R,3′R)-β,β-carotene-3,3′-diol) and/or one or more isomers and stereo-isomers (preferably mesozeaxanthin, 3R,3′S-zeaxanthin) and/or monoesters and/or diesters, preferably esters of saturated alkanoic acids, such as acetic, propionic, palmitic, stearic, and succinic acid, mono-unsaturated fatty acids, such as oleic acid, and poly-unsaturated fatty acids, such as linolic, linoleic, docosahexaenoic, and arachidonic acid, thereof;
(w) Carnosic acid and/or one or more derivatives thereof;
(x) Carnosol and/or one or more derivatives thereof;
(y) Depudecin and/or one or more derivatives thereof;
(z) Eponemycin and/or one or more derivatives thereof;
(aa) Dihydroeponemycin and/or one or more derivatives thereof;
(bb) Epoxomicin and/or one or more derivatives thereof;
(cc) Ergosterol and/or one or more derivatives thereof;
(dd) Fisetin and/or one or more derivatives thereof;
(ee) Fumagillin and/or one or more derivatives thereof;
(ff) Lactacystin and/or one or more derivatives thereof;
(gg) Luteolin and/or one or more derivatives thereof;
(hh) Motuporamine C and/or one or more derivatives thereof;
(ii) Ovalicin and/or one or more derivatives thereof;
(jj) Radicicol and/or one or more derivatives thereof;
(kk) Squalamine and/or one or more derivatives thereof;
(ll) Isoliquiritin, isoliquiritigenin, liquiritigenin and/or one or more derivatives thereof;
(mm) Very-long-chain omega-3 fatty acides (eicosapentaenoic acid [C20: 5, omega-3], decosahexaenoic acid [C22: 6, omega-3], polyunsaturated ω-3 fatty acids);
(nn) Shark cartilage extract.
(oo) Glucosinolate derivatives (Methylsulfinylalkyl glucosinolates, e.g. [1-methylsulfinylmethyl glucosinolate, 2-methylsulfinylethyl glucosinolate, 3-methylsulfinylpropyl glucosinolate (glucoiberin), 4-methylsulfinylbutyl glucosinolate (glucoraphanin), 5-methylsulfinylpentyl glucosinolate (glucoalysin), 6-methylsulfinylhexyl glucosinolate, 7-methylsulfinylheptyl glucosinolate, 8-methylsulfinyloctyl glucosinolate, 9-methylsulfinylnonyl glucosinolate, 10-methylsulfinyldodecyl glucosinolate] or allyl glucosinolate (sinigrin) or phenylethyl glucosinolate (gluconasturtiin) or 3-butenyl glucosinolate (gluconapin) or indol-3-ylmethyl glucosinolate (glucobrassicin) or derivatives thereof [N-methoxyindol-3-ylmethyl glucosinolate (neoglucobrassicin), 4-hydroxyindol-3-ylmethyl glucosinolate (4-OH glucobrassicin), 4-methoxyindol-3-ylmethyl glucosinolate (4-CH₃O glucobrassicin)]).
(pp) Isothiocyanate derivatives (Methylsulfinylalkyl isothiocyanate (1-methylsulfinylmethyl isothiocyanate, 2-methylsulfinylethyl isothiocyanate, 3-methylsulfinylpropyl isothiocyanate, 4-methylsulfinylbutyl isothiocyanate (sulforaphane), 5-methylsulfinylpentyl isothiocyanate, 6-methylsulfinylhexyl isothiocyanate (6-HITC), 7-methylsulfinylheptyl isothiocyanate, 8-methylsulfinyloctyl isothiocyanate, 9-methylsulfinylnonyl isothiocyanate, 10-methylsulfinyldodecyl isothiocyanate] or allyl isothiocyanate or phenylethyl isothiocyanate (PEITC) or 3-butenyl isothiocyanate or indole-3-ylmethylisothiocyanate) or derivatives thereof (N-methoxy indole-3-ylmethylisothiocyanate, 4-hydroxy indole-3-ylmethylisothiocyanate, 4-methoxy indole-3-ylmethylisothiocyanate) or 3-Indolemethanol (Inole-3-carbinol (I3C)).

Examples of non-cancerous symptoms and pathologies, which are associated with or favored by or caused by androgen signalling, or which incidence risks are associated with androgen signalling, or symptoms and pathologies which are sensitive to a reduction of androgen signalling comprise polycystic ovary syndrome, hyperandrogenic chronic anovulation, female infertility, ovarian hyperstimulation syndrome, cystic mastitis, amenorrhea, oligomenorrhea, accumulation of abdominal fat, insulin resistance, hyperinsulinemia, type 2 diabetes mellitus, hypertension, hirsutism, feminine acne, alopecia, menstrual disorder, hyperandrogenism, SAHA syndrome (stands for seborrhea, acne, hirsutism on face, trunk and extremities and androgenetic alopecia of the scalp), congenital adrenal hyperplasia (CAH), stress induced disorders of androgen signallings and benign prostatic hyperplasia (BPH).

Of primary interest for treatment in accordance with the present invention are polycystic ovary syndrome, insulin resistance, hyperinsulinemia, type 2 diabetes mellitus, hypertension, hirsutism, feminine acne, menstrual disorder hyperandrogenism and benign prostatic hyperplasia.

For the primary and secondary prevention and coadjuvant treatment of non-cancerous symptoms and/or pathologies sensitive to/associated with androgen signalling in accordance with the present invention, lycopene is administered to the subject or a mammal in need of such treatment, i.e. humans, pets or farm animals including birds and fish, in an amount which leads to a reduction of androgen signalling. Such amount is preferably one that results in a plasma concentration of 0.01 to 6 μM (micromolar) and may be within the range of from about 0.0005 mg/kg body weight to about 5 mg/kg body weight per day.

Optionally, lycopene is administered in combination with about 0.2 mg/kg body weight to about 30 mg/kg body weight of vitamin C per day and/or about 0.01 mg/kg body weight to about 15 mg/kg body weight of vitamin E per day.

In accordance with the present invention, lycopene or the combination of lycopene with vitamin C and/or vitamin E may, furthermore, be co-administered together with one or more of following ingredients within dosage ranges set forth below in “mg/kg” or “μg/kg” or “ng/kg” meaning “mg/kg body weight” or “μg/kg body weight” or “ng/kg body weight”:

| | |
|---|---|
| Silymarin | 0.01 mg/kg to 100 mg/kg |
| Silybin or equimolar amounts | 0.01 mg/kg to 100 mg/kg |
| of derivatives | |
| Isosilybin or equimolar | 0.01 mg/kg to 100 mg/kg |
| amounts of derivatives | |
| Silydianin or equimolar | 0.01 mg/kg to 100 mg/kg |
| amounts of derivatives | |
| Silychristin or equimolar | 0.01 mg/kg to 100 mg/kg |
| amounts of derivatives | |
| Extract of Saw Palmetto | 0.01 mg/kg to 100 mg/kg |
| (lipophilic (liposterolic) | |
| berry extract containing 80 | |
| to 90 percent fatty acids) | |
| or equimolar amounts of its | |
| components mentioned above | |
| Genistein aglycone | 0.015 mg/kg to 6 mg/kg |
| Apigenin | 0.01 mg/kg to 500 mg/kg |
| Quercetin | 0.001 mg/kg to 300 mg/kg |
| Myricetin | 0.001 mg/kg to 300 mg/kg |
| Kampferol | 0.001 mg/kg to 300 mg/kg |
| Resveratrol or equimolar | 0.01 mg/kg to 1.5 mg/kg |
| amounts of derivatives | |
| Curcumin or equimolar amounts | 0.01 mg/kg to 200 mg/kg |
| of components or derivatives | |
| mentioned above | |
| Flufenamic acid | 0.01 mg/kg to 200 mg/kg |
| Geldanamycin | 0.01 mg/kg to 200 mg/kg |
| Extract of Stephania | 0.001 mg/kg to 300 mg/kg |
| hernandifolia, or equimolar | |
| amounts of its components | |
| mentioned above | |
| Extract of Myrica rubra, or | 0.001 mg/kg to 300 mg/kg |
| equimolar amounts of its | |
| components mentioned above | |
| Astaxanthin | 0.001 mg/kg to 5 mg/kg |
| β-Carotene | 0.001 mg/kg to 5 mg/kg |
| β-Cryptoxanthin | 0.001 mg/kg to 5 mg/kg |
| (−)-epigallocatechin | 0.5 mg/kg to 15 mg/kg |
| gallate (EGCG) or | |
| (−)-epicatechin gallate | |
| (ECG) or equimolar amounts | |
| of derivatives mentioned | |
| above | |
| Lutein | 0.001 mg/kg to 5 mg/kg |
| Rhizoxin | 0.001 mg/kg to 20 mg/kg |
| Palmitoyl Rhizoxin | 0.001 mg/kg to 20 mg/kg |
| Retinoic acid or equimolar | 0.001 mg/kg to 5 mg/kg |
| amounts of derivatives | |
| mentioned above | |
| All-trans Retinol | 3 μg/kg to 100 μg/kg |
| All-trans Retinyl acetate | 3.5 μg/kg to 115 μg/kg |
| All-trans Retinol palmitate | 5.5 μg/kg to 180 μg/kg |
| Vitamin D2 (Ergocalciferol) | 0.1 ng/kg to 10 μg/kg |
| Vitamin D3 (Cholecalciferol) | 0.1 ng/kg to 10 μg/kg |
| 1α,25-Dihydroxyvitamin D3 | 0.1 ng/kg to 0.5 μg/kg |
| 25-Hydroxyvitamin D3 | 0.1 ng/kg to 10 μg/kg |
| 1α,24R,25- | 0.1 ng/kg to 0.5 μg/kg |
| Trihydroxyvitamin D3 | |
| 24,25-Dihydroxyvitamin D3 | 0.1 ng/kg to 10 μg/kg |
| Zeaxanthin | 0.001 mg/kg to 5 mg/kg |
| Carnosic acid | 0.001 mg/kg to 250 mg/kg |
| Carnosol | 0.001 mg/kg to 250 mg/kg |
| Depudecin | 0.01 mg/kg to 500 mg/kg |
| Eponemycin | 0.01 mg/kg to 500 mg/kg |
| Dihydroeponemycin | 0.01 mg/kg to 500 mg/kg |
| Epoxomicin | 0.01 mg/kg to 500 mg/kg |
| Ergosterol | 0.1 mg/kg to 2000 mg/kg |
| Fisetin | 0.01 mg/kg to 500 mg/kg |
| Fumagillin | 0.1 mg/kg to 300 mg/kg |
| Lactacystin | 0.01 mg/kg to 250 mg/kg |
| Luteolin | 0.01 mg/kg to 100 mg/kg |
| Motuporamine C | 0.1 mg/kg to 500 mg/kg |
| Ovalicin | 0.1 mg/kg to 250 mg/kg |
| Radicicol | 0.1 mg/kg to 1000 mg/kg |
| Squalamine | 0.001 to 200 mg/kg |
| Isoliquiritin | 1 ng/kg to 1 mg/kg |
| Isoliquiritigenin | 1 ng/kg to 1 mg/kg |
| Very-long-chain omega-3 fatty | 0.001 g/kg to 0.05 g/kg |
| acides, e.g. eicosapentaenoic | |
| acid [C20: 5,omega-3] or | |
| equimolar amounts of very- | |
| long-chain omega-3 fatty | |
| acides mentioned above | |
| Shark cartilage extract | 0.001 g/kg to 0.1 g/kg |
| Glucosinolate derivatives e.g. | 0.01 mg/kg to 200 mg/kg |
| 4-methylsulfinylbutyl | |
| glucosinolate (glucoraphanin) | |
| or equimolar amounts of | |
| glucosinolate derivatives | |
| mentioned above | |
| Isothiocyanate derivatives or | 0.001 mg/kg to 200 mg/kg |
| I3C e.g. 4-methylsulfinylbutyl | |
| isothiocyanate (sulforaphane) | |
| or equimolar amounts of | |
| isothiocyanate derivatives | |
| mentioned above | |

Lycopene or a combination of lycopene, vitamin C and/or vitamin E, optionally together with compounds (a) to (pp) can find use in accordance with the present invention for the completion of human nutrition, nutrition of pets and farm animals, or medical treatment of subjects, especially mammals.

Said compounds may be provided as the active ingredient in compositions, preferably for enteral application, which may be solid or liquid galenical formulations, dietary compositions or animal feed compositions. Examples of solid galenical formulations are tablets, capsules (e.g. hard or soft shell gelatin capsules), pills, sachets, powders, granules and the like which contain the active ingredient together with conventional galenical carriers. Any conventional carrier material can be utilized. The carrier material can be organic or inorganic inert carrier material suitable for oral administration. Suitable carriers include water, gelatin, gum arabic, lactose, starch, magnesium stearate, talc, vegetable oils, and the like. Additionally, additives such as flavouring agents, preservatives, stabilizers, emulsifying agents, buffers and the like may be added in accordance with accepted practices of pharmaceutical compounding. They may also be used in dietary compositions which may be a food, a food premix or a fortified food or a beverage. While the individual active ingredients are suitably administered in a single composition they may also be administered in individual dosage units.

Preferably lycopene is used in accordance with the present invention together with vitamin E, or with vitamin C or with vitamin C and vitamin E. Preferred additional components are as additional active ingredients compound (a), (b), (c), (e), (f), (h), (i), (l), (m), (n), (o), (p), (q), (r), (t), (u), (v), (w), (x), (mm), (oo), (pp), more preferably the active ingredients are (a), (b), (c), (e), (f) (h), (i), (o), (q), (r), (v), (mm) and (pp).

Particularly preferred is the administration of the following active ingredients:

Lycopene, in a concentration so that the daily consumption by a human adult is in the range of from 0.25 mg/day to 50 mg/day, preferably from 0.2 mg/day to 30 mg/day; optionally in combination with

Vitamin C or its derivative, in a concentration so that the daily consumption by a human adult is in the range of from 50 mg/day to 1000 mg/day; and/or

Vitamin E or its derivative, in a concentration so that the daily consumption by a human adult is in the range of from 15 mg/day to 600 mg/day; and/or

Silymarin (extract from Silybum marianum) and/or its four main components (silybin and/or isosilybin and/or silydianin and/or silychristin), in a concentration so that the daily consumption by a human adult of Silymarin or its four main components (silybin, isosilybin, silydianin, silychristin), respectively, is in the range of from 1 mg/day to 1000 mg/day, preferably from 50 mg/day to 800 mg/day; and/or

Saw palmetto (lipophilic extract of Sabal serrulata, syn. Serenoa repens, containing phytosterols and 80 to 90 percent fatty acids) and/or its main components (lauric acid, oleic acid, myristic acid, palmitic acid, sitosterol, campesterol, stigmasterol, cycloartenol, sitostanol, campestanol) and/or derivatives thereof (long-chain fatty acyl ester, ferrulate ester, glycosides)) in a concentration so that the daily consumption by a human adult of saw palmetto or equimolar amounts of its main components is in the range of from 1 mg/day to 1000 mg/day, preferably from 50 mg/day to 250 mg/day; and/or

Genistein, in a concentration so that the daily consumption by a human adult is in the range of from 20 mg/day to 200 mg/day; and/or

Quercetin, in a concentration so that the daily consumption by a human adult is in the range of from 1 mg/day to 500 mg/day; and/or

Myricetin, in a concentration so that the daily consumption by a human adult is in the range of from 1 mg/day to 500 mg/day; and/or

Resveratrol, in a concentration so that the daily consumption by a human adult is in the range of from 5 mg/day to 50 mg/day; and/or

Curcumin (effects of Curcuma Longa) or equimolar amount of derivatives thereof (demethoxy-curcumin, bis-demethoxycurcumin, sodium curcumionate, bis-demethylcurcumin, tetrahydrocurcumin, hexahydrocurcumin, diacteylcurcumin, triethylcurcumin) and/or equimolar amount of ist main components components (curcumin (diferuloylmethane), demethoxycurcumin, bisdemethoxycurcumin) and/or derivatives thereof (glucuronides, sulfates), in a concentration so that the daily consumption by a human adult is in the range of from 10 mg/day to 1000 mg/day, preferably from 50 mg/day to 800 mg/day; and/or

β-Carotene, in a concentration so that the daily consumption by a human adult is in the range of from 0.1 mg/day to 20 mg/day, preferably from 2 mg/day to 10 mg/day; and/or

(−)-Epigallocatechin gallate (EGCG), in a concentration so that the daily consumption by a human adult is in the range of from 50 mg/day to 500 mg/day; and/or

Lutein, in a concentration so that the daily consumption by a human adult is in the range of from 0.mg/day to 50 mg/day, preferably from 0.25 mg/day to 30 mg/day; and/or

Zeaxanthin, in a concentration so that the daily consumption by a human adult is in the range of from 0.1 mg/day to 50 mg/day, preferably from 0.25 mg/day to 30 mg/day, and/or

of very-long-chain omega-3 fatty acids, e.g. eicosapentaenoic acid [C20: 5, omega-3] or equimolar amounts of very-long-chain omega-3 fatty acids, in a concentration so that the daily consumption by a human adult is in the range of from 1 mg/day to 500 mg/day, and/or

Isothiocyanate derivatives or I3C, e.g. 4-methylsulfinylbutyl isothiocyanate (sulforaphane) or equimolar amounts of isothiocyanate derivatives mentioned above, in a concentration so that the daily consumption by a human adult is in the range of from 0.1 mg/day to 50 mg/day, preferably from 0.25 mg/day to 30 mg/day.

Typical examples of galenical formulations for use in accordance with the present invention are given below. The Examples are for the purpose of illustrating the invention and are not intended to limit the scope of the invention in any way.

The following Examples illustrate the invention further.

### EXAMPLE 1

A tablet for the coadjuvant treatment of feminine acne is formulated to contain 5 mg of lycopene, 200 mg of vitamin E, 250 mg of vitamin C, 37.5 mg of resveratrol. The daily dose corresponds to said amounts in form of two tablets with half of said amounts each.

### EXAMPLE 2

A tablet for the prevention of polycystic ovary syndrome is formulated to contain 2.5 mg of lycopene, 250 mg of vitamin E, 100 mg of vitamin C, 100 mg silymarin.

1. A method of manufacture of a composition for the primary and secondary prevention, incidence risk reduction, coadjuvant treatment or treatment of non-cancerous symptoms and/or pathologies, which are associated with, favored by or caused by androgen signalling, or which are sensitive to a reduction of androgen signalling comprising admixing an effective amount lycopene with a conventional carrier material.

2. A method according to claim 1 wherein the composition comprises lycopene in combination with vitamin E.

3. A method according to claim 1 wherein the composition comprises of lycopene in combination with vitamin E and/or vitamin C.

4. A method according to claim 1 wherein the composition comprises lycopene in combination with one or more compounds selected from: silymarin, silybin or equimolar amounts of derivatives, isosilybin or equimolar amounts of derivatives, silydianin or equimolar amounts of derivatives, silychristin or equimolar amounts of derivatives, saw palmetto or equimolar amounts of derivatives, free fatty acids (lauric acid, oleic acid, myristic acid, palmitic acid) or equimolar amounts of derivatives, phytosterols (sistosterol, campesterol, stigmasterol, cycloartenol, sitostanol, campestanol) or equimolar amounts of derivatives (long-chain fatty acid acyl ester, ferrulate ester, glycosides), genistein aglycone, genistein glucosides, genistein sulfates, genistein glucuronides, apigenin, quercetin or equimolar amounts of derivatives (quercetine glucosides, quercetin glucuronides, quercetine sulphates, methylquercetin (isohamnetin 3′-O-methylquercetin), tamarixetin(4′-O-methylquercetin)), myricetin, kampferol, resveratrol or equimolar amounts of derivatives, Curcumin (effects of Curcuma Longa) or equimolar amount of derivatives thereof (demethoxy-curcumin, bis-demethoxycurcumin, sodium curcumionate, bis-demethylcurcumin, tetrahydrocurcumin, hexahydrocurcumin, diacteylcurcumin, triethylcurcumin) and/or equimolar amount of ist main components (curcumin (diferuloylmethane), demethoxycurcumin, bisdemethoxycurcumin) and/or derivatives thereof (glucuronides, sulfates), flufenamic acid, geldanamycin, extract of Stephania hemandifolia and/or one or more of its components (e.g. 4-demethylhasubanonine, epistephanine), extract of Myrica rubra and/or one or more derivatives thereof and/or one or more of its components being diarylheptanoids (Quercetin, myricanone, myricanol, and myricetin) named acerogenin and their glycosides named aceroside and/or derivatives thereof, astaxanthin, β-carotene, β-cryptoxanthin, (−)-epigallocatechin gallate (EGCG) or (−)-epicatechin gallate (ECG) or equimolar amounts of derivatives, lutein, rhizoxin, palmitoyl rhizoxin, all-trans retinol, retinoic acids (all-trans retinoic acid and/or 13-cis retinoic acid and/or 9-cis retinoic acid) and/or one or more derivatives thereof (4-hydroxyphenylretinamide or retinyl esters such as all-trans retinyl acetate); all-trans retinyl acetate, all-trans retinol palmitate, vitamin D2 (ergocalciferol), vitamin D3, (cholecalciferol), 1 α,25-dihydroxyvitamin D3,25-hydroxyvitamin D3,1α,24R,25-trihydroxyvitamin D3,24,25-dihydroxyvitamin D3, zeaxanthin, carnosic acid, carnosol, depudecin, eponemycin, dihydroeponemycin, epoxomicin, ergosterol, fisetin, fumagillin, lactacystin, luteolin, motuporamine C, ovalicin, radicicol, squalamine, isoliquiritin, isoliquiritigenin, very-long-chain omega-3 fatty acids (eicosapentaenoic acid [C20: 5, omega-3], decosahexaenoic acid [C22: 6, omega-3], polyunsaturated ω-3 fatty acids), shark cartilage extract, glucosinolate derivatives (Methylsulfinylalkyl glucosinolates (1-methylsulfinylmethyl glucosinolate, 2-methylsulfinylethyl glucosinolate, 3-methylsulfinylpropyl glucosinolate (glucoiberin), 4-methylsulfinylbutyl glucosinolate (glucoraphanin), 5-methylsulfinylpentyl glucosinolate (glucoalysin), 6-methylsulfinylhexyl glucosinolate, 7-methylsulfinylheptyl glucosinolate, 8-methylsulfinyloctyl glucosinolate, 9-methylsulfinylnonyl glucosinolate, 10-methylsulfinyidodecyl glucosinolate) or allyl glucosinolate (sinigrin) or indol-3-ylmethyl glucosinolate (glucobrassicin) or derivatives thereof (N-methoxyindol-3-ylmethyl glucosinolate (neoglucobrassicin), 4-hydroxyindol-3-ylmethyl glucosinolate (4-OH glucobrassicin), 4-methoxyindol-3-ylmethyl glucosinolate (4-CH₃O glucobrassicin)) or phenylethyl glucosinolate (gluconasturtiin) or 3-butenyl glucosinolate (gluconapin)), or isothiocyanate derivatives (Methylsulfinylalkyl isothiocyanate (1-methylsulfinylmethyl isothiocyanate, 2-methylsulfinylethyl isothiocyanate, 3-methylsulfinylpropyl isothiocyanate, 4-methylsulfinylbutyl isothiocyanate (sulforaphane), 5-methylsulfinylpentyl isothiocyanate, 6-methylsulfinylhexyl isothiocyanate (6-HITC), 7-methylsulfinylheptyl isothiocyanate, 8-methylsulfinyloctyl isothiocyanate, 9-methylsulfinylnonyl isothiocyanate, 10-methylsulfinyldodecyl isothiocyanate) or allyl isothiocyanate, indole-3-ylmethylisothiocyanate, N-methoxy indole-3-ylmethylisothiocyanate, 4-hydroxy indole-3-ylmethylisothiocyanate, 4-methoxy indole-3-ylmethylisothiocyanate, 3-Indolemethanol, phenylethyl isothiocyanate (PEITC), 3-butenyl isothiocyanate).

5. A method according to claim 1, in which said symptom or pathology is polycystic ovary syndrome, hyperandrogenic chronic anovulation, female infertility, ovarian hyperstimulation syndrome, amenorrhea, oligomenorrhea, accumulation of abdominal fat, insulin resistance, hyperinsulinemia, type 2 diabetes mellitus, hypertension, hirsutism, feminine acne, alopecia, menstrual disorder, hyperandrogenism, SAHA syndrome, congenital adrenal hyperplasia (CAH), stress induced imbalance of androgen signalling or benign prostatic hyperplasia.

6. A method according to claim 5, in which said symptom or pathology is polycystic ovary syndrome, hyperandrogenic chronic anovulation, female infertility, ovarian hyperstimulation syndrome, amenorrhea, oligomenorrhea, accumulation of abdominal fat, insulin resistance, hyperinsulinemia, hirsutism, feminine acne, alopecia, menstrual disorder, hyperandrogenism, SAHA syndrome, congenital adrenal hyperplasia (CAH) or benign prostatic hyperplasia.

7. A method according to claim 6, in which said symptom or pathology is polycystic ovary syndrome, obesity, insulin resistance, hyperinsulinemia, type 2 diabetes mellitus, hypertension, hirsutism, feminine acne, menstrual disorder, hyperandrogenism or benign próstatic hyperplasia.

8. A method according to claim 7, in which said pathology is feminine acne.

9. A method according to claim 7, in which said pathology is hirsutism.

10. A method according to claim 7, in which said pathology is type 2 diabetes mellitus.

11. A method according to claim 7, in which said pathology is polycystic ovary syndrome.

12. A method according to claim 7, in which said pathology is benign prostatic hyperplasia.

13. A method according to claim 1 wherein the composition is a solid or liquid galenical formulation, a dietary composition or an animal feed composition.

14. A method according to claim 13 wherein a dosage unit of said solid galenical formulation contains from about 0.25 mg to about 50 mg of lycopene.

15. A method according to claim 14 wherein a dosage unit of said solid galenical formulation further contains from about 10 mg to about 1000 mg of vitamin E.

16. A method according to claim 14 wherein a dosage unit of said solid galenical formulation further contains from about 50 mg to about 1000 mg of vitamin C.

17. A method according to claim 14 wherein the composition comprises lycopene in combination with vitamin E, vitamin C and resveratrol in the manufacture of a solid galenical formulation for the coadjuvant treatment of feminine acne.

18. A method according to claim 14 wherein the composition is in the form of a solid galenical formulation for the prevention of polycystic ovary syndrome containing lycopene in combination with vitamin E, vitamin C, silymarin in one dosage unit.

19. A method according to claim 13 wherein said liquid galenical formulation contains from about 0.1 mg to about 100 mg of lycopene per ml.

20. A method according to claim 17 wherein said liquid galenical formulation further contains from about 10 mg to about 300 mg of vitamin E per ml.

21. A method according to claim 20 wherein said liquid galenical formulation contains from about 50 mg to about 100 mg of vitamin C per ml.

22. A method according to claim 13 wherein said dietary composition or animal feed composition contains from about 0.025 mg to about 5 mg of lycopene per g.

23. A method according to claim 21 wherein said dietary composition or animal feed composition further contains from about 1.5 mg to about 30 mg of vitamin E per g.

24. A method according to claim 21 wherein said dietary composition or animal feed composition further contains from about 5 mg to about 50 mg of vitamin C per g.

25. A method of prevention or treatment of symptoms or pathologies associated with androgen signalling, which comprises administering to a subject (mammal or non-mammal, human or pet including birds and fish, or mammal or non-mammal farm animal) in need of such treatment for therapy or prophylaxis an effective amount of lycopene.

26. A method as in claim 25 wherein about 0.25 mg to about 50 mg of lycopene are administered per day to a human adult.

27. A method as in claim 26 wherein about 1 mg to about 30 mg of lycopene are administered per day to a human adult.

28. A method as in claim 25 wherein, additionally, about 15 mg to about 600 mg of vitamin E are administered per day to a human adult.

29. A method as in claim 25 wherein, additionally, about 50 to about 1000 mg of vitamin C are administered per day to a human adult.

30. A method of treating non-cancerous symptoms and/or pathologies sensitive to lycopene which comprises administering to a mammal, mammal or non-mammal pets including birds and fish, or mammal or non-mammal farm animal in need of such treatment an amount of lycopene which leads to a reduction of androgen signalling.

31. The method according to claim 30, wherein an amount of lycopene is administered which results in a plasma concentration of 0.01 to 6 μM.
